(19) United States
(12) Patent Application Publication
(10) Pub. No.: US 2023/0151410 A1
(43) Pub. Date: May 18, 2023

(54) Identifying Feline Renal Risk Factors and Compositions and Methods for Improving Feline Health

(71) Applicant: Hill's Pet Nutrition, Inc., Topeka, KS (US)

(72) Inventors: Jeffrey BROCKMAN, Lawrence, KS (US); Eden Ephraim, Lawrence, KS (US)

(73) Assignee: Hill's Pet Nutrition, Inc., Topeka, KS (US)

(21) Appl. No.: 17/931,360

(22) Filed: Sep. 12, 2022

Related U.S. Application Data
(60) Provisional application No. 63/264,264, filed on Nov. 18, 2021.

(51) Int. Cl. C12Q 1/6827; C12Q 1/6874; C12Q 1/6837; G01N 33/49

(52) U.S. Cl. CPC C12Q 1/6827; C12Q 1/6874; C12Q 1/6837; G01N 33/492; G01N 2800/347

(57) ABSTRACT

Methods of detecting the presence of one copy or 2 copies of a minor allele of one or more single nucleotide polymorphisms selected from the group consisting of SNP A3_117040611, SNP A3_117041908 and SNP A3_117081913 are disclosed. Methods of identifying a feline subject as being at an increased likelihood of developing cat kidney disease are disclosed. Methods of monitoring the health of a feline subject identified as having an increased likelihood of developing CKD are provided. Methods of monitoring the health of a feline subject and response to treatment for renal disease that comprise quantifying at 2PY present are provided. Methods of delaying onset and severity of cat kidney disease in a feline subject identified as having an increased likelihood of developing CKD and methods of treating cats that have CKD are provided.

### CROSS-REFERENCE TO RELATED APPLICATION

This application claims the benefit of U.S. Provisional Application No. 63/264,264, filed Nov. 18, 2022, which is incorporated by reference herein in its entirety.

### REFERENCE TO AN ELECTRONIC SEQUENCE LISTING

The application contains a Sequence Listing which has been submitted electronically in XML format. The contents of the electronic sequence listing Named: 14003-00-US-01-HL_SEQ_Listing_ST26.xml; Size: 6,455 bytes; and Date of Creation: Sep. 9, 2022, is herein incorporated by reference in its entirety.

### BACKGROUND

Reagents, kits and methods for evaluating the genetic makeup of a cat are provided for identifying cats at increased risk of developing cat kidney disease (CKD). Reagents, kits and methods are provided for monitoring the presence of biomarkers in cats that have been identified as being at increased risk of developing CKD. Compositions for and methods of preventing, reducing the severity and/or delaying the onset of CKD in cats identified as being at increased risk of developing CKD are provided. Reagents, kits and methods are provided for monitoring responsiveness of cats undergoing such methods of preventing, reducing the severity and/or delaying the onset of CKD in cats identified as being at increased risk of developing CKD.

Chronic renal disease (chronic kidney disease, CKD) is a common affliction of aging cats with a prevalence rate of 1-3% in all cats and up to 80% of geriatric cats. CKD can progress quietly for years before overt clinical signs are noted. Annual screening of healthy cats may be useful to establish a baseline which can be monitored for changes over time. CKD screening may include a physical examination, blood testing, and urinalysis. Physical examination may include body and muscle condition scoring, kidney palpation and fundic examination. A complete blood count can help detect anemia, which is commonly mild and nonregenerative with CKD. Two biochemical byproducts in the bloodstream, blood urea nitrogen (BUN) and creatinine, which are used in diagnosing CKD, are measured during screening. Measurement of serum symmetric dimethylarginine (SDMA) concentrations in blood may also be useful as part of comprehensive screening procedure. Urinalysis, includes measurement of urine specific gravity (USpG) as well as microscopic sediment examination to determine the presence or absence of proteinuria, red and white blood cells, bacteria, crystals, and casts. Urinalysis is a particularly useful screening tool since loss of concentrating ability often occurs before other indicators of CDK can be detected.

The International Renal Interest Society (IRIS) provides guidelines for diagnosing, staging and treating feline CDK.

Feline CDK may be diagnosed using clinical observations, physical examination findings, blood test results and urinalysis. Clinical signs of CDK include polyuria, polydipsia, weight loss, decreased appetite, lethargy, dehydration, vomiting, and bad breath. Physical examination finding for cats with CDK may include palpable kidney abnormalities, evidence of weight loss, dehydration, pale mucous membranes, uremic ulcers, evidence of hypertension, i.e., retinal hemorrhages/detachment. After overt signs of CKD are present, some degree of azotemia (increased BUN and creatinine) will be detected. Varying degrees of hyperphosphatemia will also be present and often worsen as CKD progresses. Creatinine levels increasing within the reference interval and/or SDMA levels increasing within the reference interval, persistent increased SDMA >14 μg/dL, abnormal kidney imaging, persistent renal proteinuria showing a urine protein to creatinine (UPC) ratio >0.4 in cats are consistent with a CKD diagnosis. Increased creatinine and SDMA concentrations plus USpG of <1.035 indicate a more advanced CKD diagnosis.

IRIS categorizes CKD into 4 stages:
Stage 1—nonazotemic (creatine levels are normal);
Stage 2—mild azotemia (creatine levels are normal or mildly elevated);
Stage 3—moderate azotemia; and
Stage 4—severe azotemia.

In Stage 1 feline CDK, stable creatinine levels are less than 1.6 mg/dL (140 μmon) and stable SDMA levels are less than 18 μg/dL. In Stage 2 feline CDK, stable creatinine levels are 1.6-2.8 mg/dL (140-250 μmon) and stable SDMA levels are 18-25 μg/dL. In Stage 3 feline CDK, stable creatinine levels are 2.9-5.0 mg/dL (251-440 μmon) and stable SDMA levels are 26-38 μg/dL. In Stage 4 feline CDK, stable creatinine levels are greater than 5.0 mg/dL (440 μmon) and stable SDMA levels are greater than 38 μg/dL. Each stage of feline CDK may be sub-staged based on proteinuria and blood pressure. A UPC ratio of <0.2 is nonproteinuric, a UPC ratio of 0.2-0.4 is borderline proteinuric, and a UPC ratio of >0.4 is proteinuric. A systolic blood pressure of <140 mm Hg is normotensive, a systolic blood pressure of 140-159 mm Hg is prehypertensive, a systolic blood pressure of 160-179 mm Hg is hypertensive, and a systolic blood pressure of ≥180 mm Hg are severely hypertensive.

Treatment recommendations for cats with Stage 1 CKD are as follows: nephrotoxic drugs should be used with caution; prerenal and postrenal abnormalities should be corrected; fresh water should be available at all times; trends in creatinine and SDMA levels should be monitored to document stability or progression; investigate for and treat possible underlying disease and/or complications; hypertension should be treated if systolic blood pressure is persistently >160 mm Hg or evidence of end-organ damage; persistent proteinuria (UPC >0.4) should be treated with renal therapeutic diet and medication; phosphorus should be kept at <4.6 mg/dL (<1.5 mmol/L); and, if required, renal therapeutic diet plus phosphate binder should be used. Treatment recommendations for cats with Stage 2 CKD are the same as the treatment for Stage 1 CKD but a renal therapeutic diet should be used and hypokalemia should be treated. Treatment recommendations for cats with Stage 3 CKD are the same as the treatment for Stage 2 CKD but phosphorus should be kept at <5.0 mg/dL (<1.6 mmol/L); metabolic acidosis should be treated; treatment of anemia should be considered; vomiting, inappetence, and nausea should be treated; and increased enteral or subcutaneous fluids may be required to maintain hydration. Treatment recommendations for cats with Stage 4 CKD are the same as the treatment for Stage 3 CKD but phosphorus should be kept at <6.0 mg/dL (<1.9 mmol/L); and a feeding tube for nutritional and hydration support and ease of medicating should be considered.

Age, sex, breed predispositions, relevant historical information, including medication history, toxin/toxicant exposure, and diet provided limited insight in determining risk for developing CKD. There is currently no testing available to assess risk, determine if any preventative interventions, such as a modified diet, are indicated and monitor response to such interventions.

There is a need to develop improved methods to identify cats having increased likelihood or risk of developing CKD. There is a need to develop methods to determine if a cat has a relatively high, intermediate or low likelihood or risk of developing CKD. There is a need for improved compositions and methods that reduce the risk of developing CKD, including compositions and methods that reduce the risk of developing CKD in at least a subpopulation of cats having genetic markers or phenotypic traits. There is a need for improved compositions and methods that prevent or delay onset of CKD in at least a subpopulation of cats having genetic markers or phenotypic traits. There is a need to develop methods to identify cats that will benefit from treatment to reduce the likelihood or risk of developing CKD. There is a need to develop methods to monitor responsiveness to treatment of cats identified as having an increased likelihood or risk of developing CKD. There is a need for kits, reagents, other articles, and compositions useful in such methods.

### BRIEF SUMMARY

Methods of detecting the presence of one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject are provided. The methods comprise obtaining a biological sample from the feline subject and analyzing a biological sample obtained from the feline subject to detect one copy or 2 copies of a minor allele of one or more single nucleotide polymorphisms selected from the group consisting SNP A3_117040611, SNP A3_117041908 and SNP A3_117081913 and a single nucleotide polymorphism in linkage disequilibrium with one or more thereof.

Methods of identifying a feline subject as being at an increased likelihood of developing cat kidney disease are provided. The methods comprise analyzing a biological sample obtained from the feline subject for the presence of one copy or 2 copies of a minor allele of two or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject; wherein the presence of one copy or 2 copies of the minor allele of two or more single nucleotide polymorphisms selected from the group consisting of SNP A3_117040611, SNP A3_117041908 and SNP A3_117081913 indicates that the feline subject has an increased likelihood of developing cat kidney disease within its lifetime.

Methods of identifying a feline subject as being at an increased likelihood of developing cat kidney disease are provided that comprise analyzing a biological sample obtained from the feline subject for the presence of one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject by performing DNA sequencing, restriction enzyme digest, polymerase chain reaction (PCR), hybridization, real-time PCR, reverse transcriptase PCR, or ligase chain reaction. The presence of one copy or 2 copies of the minor allele of one or more single nucleotide polymorphisms selected from the group consisting of SNP A3_117040611, SNP A3_117041908 and SNP A3_117081913 indicates that the feline subject is at an increased likelihood of for developing cat kidney disease within its lifetime.

Methods of identifying a feline subject as being at an increased likelihood of developing cat kidney disease are provided that comprise analyzing a biological sample obtained from the feline subject for the presence of one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject wherein the sample is analyzed by performing at least one nucleic acid analysis technique selected from: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis, heteroduplex analysis, chemical mismatch cleavage (CMC), RNase protection assays, use of polypeptides that recognize nucleotide mismatches, allele-specific PCR, sequence analysis, and SNP genotyping. The presence of one copy or 2 copies of the minor allele of one or more single nucleotide polymorphisms selected from the group consisting of SNP A3_117040611, SNP A3_117041908 and SNP A3_117081913 indicates that the feline subject is at an increased likelihood of for developing cat kidney disease within its lifetime.

Methods of identifying a feline subject as being at an increased likelihood of developing cat kidney disease are provided that comprise analyzing a biological sample obtained from the feline subject for the presence of one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject wherein the sample is analyzed by performing hybridization-based methods, enzyme-based methods, post-amplification methods based on physical properties of DNA, and sequencing methods. The presence of one copy or 2 copies of the minor allele of one or more single nucleotide polymorphisms selected from the group consisting of SNP A3_117040611, SNP A3_117041908 and SNP A3_117081913 indicates that the feline subject is at an increased likelihood of for developing cat kidney disease within its lifetime.

Methods of identifying a feline subject as being at an increased likelihood of developing cat kidney disease are provided that comprise analyzing a biological sample obtained from the feline subject for the presence of one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject wherein the sample is analyzed by performing hybridization-based methods selected from the group consisting of dynamic allele-specific hybridization, molecular beacon methods and SNP microarrays; enzyme-based methods selected from the group consisting of restriction fragment length polymorphism (RFLP), PCR-based methods, Flap endonuclease, primer extension methods, 5′-nuclease and oligonucleotide ligation assay; post-amplification methods based on physical properties of DNA selected from the group consisting of single strand conformation polymorphism, temperature gradient gel electrophoresis, denaturing high performance liquid chromatography, high-resolution amplicon melting, DNA mismatch-binding proteins, SNPlex, and surveyor nuclease assay; and sequencing methods. The presence of one copy or 2 copies of the minor allele of one or more single nucleotide polymorphisms selected from the group consisting of SNP A3_117040611, SNP A3_117041908 and SNP A3_117081913 indicates that the feline subject is at an increased likelihood of for developing cat kidney disease within its lifetime.

Methods of monitoring the health of feline subject identified as having an increased likelihood of developing CKD are provided. The methods comprise identifying a feline subject as being at an increased likelihood of developing cat kidney disease according and quantifying at two or more time points the level of 2PY present in a blood sample from the feline wherein an elevation of 2PY levels over time indicates that the feline subject is developing kidney disease.

Methods of monitoring the health of feline subject are provided. The methods comprise quantifying at two or more time points the level of 2PY present in a blood sample from the feline subject identified as having cat kidney disease wherein an elevation of 2PY levels over time indicates that the feline subject is developing kidney disease.

Methods of delaying onset and severity of cat kidney disease in a feline subject identified as having an increased likelihood of developing CKD are provided. The methods comprise detecting in a biological sample from the feline subject, the presence of one copy or 2 copies of a minor allele of one or more single nucleotide polymorphisms selected from SNP A3_117040611, SNP A3_117041908 and SNP A3_117081913; and administering to the feline subject a composition comprising an effective amount of: betaine, oat beta glucan and one or more of short-chain fructo-oligosaccharide and apple pomace.

Methods of delaying onset and severity of cat kidney disease in a feline subject suspected of having an increased likelihood of developing CKD are provided. The methods comprise administering to the feline subject a composition comprising an effective amount of: betaine, oat beta glucan and one or more of short-chain fructo-oligosaccharide and apple pomace.

Methods of monitoring the treatment of feline subject identified as having cat kidney disease and being treated for cat kidney disease are provided. The methods comprise quantifying the level of 2PY present in a blood sample from the feline subject identified as having cat kidney disease wherein the blood sample was obtained prior to treating the feline subject for cat kidney disease, administering treatment to the feline subject and quantifying the level of 2PY present in a blood sample from the feline subject obtained after treating the feline subject for cat kidney disease. A reduction of 2PY levels after treatment indicates that the feline subject is responding to the treatment.

### BRIEF DESCRIPTION OF THE FIGURES

FIG. 1 shows data from an analysis of 2PY levels in healthy cats and cats diagnosed as having renal disease. Cats diagnosed with renal disease have >3 fold higher levels of 2PY.

FIG. 2 shows a Manhattan plot of data from QTL analysis of 2PY in cats.

FIG. 3 shows relative plasma levels of 2PY and creatinine after cats consumed baseline food, Diet 1 or Diet 2, each for 30 days.

### DETAILED DESCRIPTION

The following description of the preferred embodiment(s) is merely exemplary in nature and is in no way intended to limit the invention, its application, or uses.

As used herein and in the appended claims, the singular forms “a”, “an”, and “the” include plural reference unless the context clearly dictates otherwise.

The term “cat” includes those cats which are companion animals known as domestic cats or house cats, or Felis domesticus. The term cat is synonymous with the term feline.

A “food,” “food composition,” “pet food composition” or “cat food composition” can, in some embodiments, be a nutritionally complete diet for cat to which it is fed.

“Daily nutritional intake” and “total nutritional intake per day” refer to dry matter intake per day. That is, water weight is not included in calculating the amount of nutrition consumed per day. To the extent that food and food ingredient contain water/moisture, the dry matter represents everything in the sample other than water including protein, fiber, fat, minerals, etc. Dry matter weight is the total weight minus the weight of any water. Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day. The skilled artisan would readily recognize and understand nutritional amounts and percentages expressed as dry matter amounts, dry matter weights and dry matter percentages. Since foods, whether wet, moist or dry, generally contain as certain amount of water, when calculating daily dry matter intake, the water component of such food is excluded. To calculate total daily nutritional intake, which is dry matter intake per day, water is excluded. To calculate percent of an ingredient of total daily intake on a dry matter basis, water is removed from the total intake to give total daily dry matter intake and the percent of the ingredient is based on amount of ingredient present as dry matter.

As used herein, an “ingredient” refers to any component of a composition.

As used herein, the term “treatment” refers to eliminating, reducing the severity or preventing one or more symptoms.

The term “nutrient” refers to a substance that provides nourishment. In some cases, an ingredient may comprise more than one “nutrient,” for example, a composition may comprise corn comprising important nutrients including both protein and carbohydrate.

Food compositions can be provided to in the form of cat food. A variety of commonly known types of cat foods are available to cat owners. The selection of cat food includes but is not limited to wet cat food, semi-moist cat food, dry cat food and cat treats. Wet cat food generally has a moisture content greater than about 65%. Semi-moist cat food typically has a moisture content between about 20% and about 65% and may include humectants, potassium sorbate, and other ingredients to prevent microbial growth (bacteria and mold). Dry cat food such as but not limited to food kibbles generally has a moisture content below about 15%. Pet treats typically may be semi-moist, chewable treats; dry treats in any number of forms, or baked, extruded or stamped treats; confection treats; or other kinds of treats as is known to one skilled in the art.

As used herein, the term “kibble” or “food kibble” refers to a particulate pellet like component of cat feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

As used herein, the term “extrude” or “extrusion” refers to the process of sending preconditioned and/or prepared ingredient mixtures through an extruder. In some embodiments of extrusion, food kibbles are formed by an extrusion processes wherein a kibble dough, including a mixture of wet and dry ingredients, can be extruded under heat and pressure to form the food kibble. Any type of extruder can be used, examples of which include but are not limited to single screw extruders and twin-screw extruders. The list of sources, ingredients, and components as described hereinafter are listed such that combinations and mixtures thereof are also contemplated and within the scope herein.

As contemplated herein, compositions are meant to encompass, but not be limited to, nutritionally-complete and balanced cat food compositions. A “nutritionally complete diet” is a diet that includes sufficient nutrients for maintenance of normal health of a healthy cat on the diet. Nutritionally complete and balanced cat food compositions are familiar to one of skill in the art. For example, substances such as nutrients and ingredients suitable for nutritionally complete and balanced animal feed compositions, and recommended amounts thereof, may be found for example, in the Official Publication of the Association of American Feed Control Officials, Inc. (AAFCO), Atlanta, Ga., (2012).

As used herein, the term “supplement(s)” include, but are not limited to, a feed used with another feed to improve nutritive balance or performance of the total diet for an animal. Supplements include, but are not limited to, compositions that are fed undiluted as a supplement to other feeds, offered free choice with other parts of an animal's ration that are separately available, or diluted and mixed with an animal's regular feed to produce a complete feed. The AAFCO guidelines, for example, contain a discussion relating to supplements in the Official Publication of the Association of American Feed Control Officials, Inc. (AAFCO), Atlanta, Ga. (2012). Supplements may be in various forms including, for example, powders, liquids, syrups, pills, encapsulated compositions and the like.

As described herein, increased or elevated likelihood or risk of developing CKD refers to having a greater than average chance that an individual cat will develop CKD compared to that of a cat that has a heterozygous genotype of the major alleles for the specific SNPs discussed herein.

2PY

As described herein, 2PY can be used as a biomarker for renal disease in cats. In humans, N-methyl-2-pyridone-5-carboxamide (2PY), which is a metabolite of nicotinic acid (Niacin, Vitamin B3), has been designated as a uremic toxin which additionally may exacerbate kidney tissue damage through its inhibition of poly ADP-ribose polymerase 1 (PARP-1). PARP-1 is thought to detect DNA damage and initiate DNA repair mechanisms and its inhibition may lead to apoptosis and cell death. Nicotinamide is the water-soluble amine derivative of nicotinic acid. Nicotinamide is methylated to N-methyl-nicotinamide which is then converted into either 2PY or N-methyl-4-pyridone-5-carboxamide (4PY) by aldehyde oxidase (AOX) (EC Number 1.2.3.1).

Data in Example 1 below established that 2PY can serve as a biomarker for disease. 2PY levels were found to be elevated in cats having CKD. Accordingly, measuring 2PY levels in cats that present as being healthy, including those considered to be at a higher risk for CKD, can be performed as part of a general screening procedure. Measured 2PY levels in such cats can be compared to a standard level that is representative of 2PY levels in cats without disease and a finding of elevated 2PY levels indicates that the cat is likely to have renal disease. 2PY can also be used to evaluate cats suspected as having renal disease, i.e., demonstrating some symptoms of renal disease as well as to confirm the presence of disease in cats who have been diagnosed as having renal disease. Additionally, 2PY has been found in short term studies to be more sensitive than the commonly used biomarker, creatinine, for demonstrating efficacy of dietary and other interventions. Thus, 2PY allows for improved ability to monitor response to treatment as well as to improve the ability to develop more efficacious interventions at a faster pace. Two different formulations have been developed that effectively reduce the 2PY levels of both normal and renal cats.

Levels of 2PY may be measured in blood samples. Blood is collected in order to determine plasma metabolomic profiles. Extracted supernatant is split and run on gas chromatography and liquid chromatography mass spectrometer platforms. The peak for 2PY is known and the area under the peak for each sample can be normalized to a known sample (See also: Evans, A. M., et al. (2009). Integrated, nontargeted ultrahigh performance liquid chromatography/electrospray ionization tandem mass spectrometry platform for the identification and relative quantification of the small-molecule complement of biological systems. Anal. Chem. 81, 6656-6667.) Gas chromatography (for hydrophobic molecules) and liquid chromatography (for hydrophilic molecules) are used to identify and provide relative quantification of metabolites such as 2PY present in plasma samples. (See also: Ballet, C. et al. (2018) New enzymatic and mass spectrometric methodology for the selective investigation of gut microbiota-derived metabolites, Chem. Sci. 9, 6233-6239; Akiyama, Y et al. (2012) A Metabolomic Approach to Clarifying the Effect of AST-120 on 5/6 Nephrectomized Rats by Capillary Electrophoresis with Mass Spectrometry (CE-MS) Toxins 4(11):1309-1322; and Kikuchi K, et al. (2010) Metabolomic search for uremic toxins as indicators of the effect of an oral sorbent AST-120 by liquid chromatography/tandem mass spectrometry. J Chromatogr B Analyt Technol Biomed Life Sci 878:2997-3002.) Commercial laboratories such as Metabolon, (Durham, N.C., USA) measured levels of 2PY in plasma. In some embodiments, the test samples are evaluated with one or more known samples containing a known amount of 2PY and the results of the test sample are normalized with respect to those for the known sample. In some embodiments, the known samples contain 2PY at a level which represents a normal range. A standard may be established based upon data from healthy cats which is used to inform the results with respect to the relative quantity of 2PY in the test sample to the 2PY in the known sample. The standard provides a cut off wherein that is used to determine if the quantity of 2PY in the test sample is indicative of the presence or absence of disease. In some embodiments, a positive control sample may be provided which contains 2PY at a level which represents a disease level.

Genetic Markers

Two independent genetic markers have been identified that predict approximately 25-30% of cats that produce higher than average levels of 2PY. These genetic markers are single nucleotide polymorphisms (SNPs). They can be used to identify cats that are at a higher risk for a faster progression of renal disease and therefore benefit the most from a dietary intervention that reduces 2PY levels.

Single nucleotide polymorphisms (SNPs), a common type of genetic variation, are single base pair mutations at a specific locus. That is, a SNP is a difference in a single nucleotide in a DNA sequence that occurs at a specific position in a genome. Typically, for a SNP at a specific position, there are two possible nucleotide variations, which are referred to as alleles for that position. Within a population, the nucleotide variation that most commonly appears at a specific base position in a genome is referred to as the major allele; the nucleotide variation that is less common at that specific base position is referred to as the minor allele. Felines, like most multicellular organisms have two sets of chromosomes. Thus, each cat has two copies of each gene or locus and therefore two copies of each SNP. Accordingly, for each SNP in the cat's genome, the cat may have two copies of the major allele, or one copy of the minor allele and one copy of the minor allele, or two copies of the minor allele.

When a particular allele occurs disproportionately among individuals with a disease or condition relative to its occurrence in a broader population, SNPs can act as biological markers for likelihood or risk of developing various particular diseases. SNP genotyping refers to identification of the alleles of a SNP present within the genome. There are numerous methods for detecting SNPs and performing SNP genotyping.

Genetic association studies identified genetic markers that can be used to identify cats as being at a higher likelihood, increased risk for developing CKD. In an investigation of cats with elevated 2PY set forth in the Example 2 below, two SNPs have been determined to be associated with the location of the AOX gene in cats in which the presence of minor alleles occurs disproportionately in cats with CKD. As noted above, AOX converts N-methyl-nicotinamide into 2PY or 4PY. The gene for AOX is located on chromosome Cl in cat. Xanthine Dehydrogenase/Oxidase gene (XDH) is a bifunctional enzyme with dehydrogenase activity (EC Number 1.17.1.4) and aldehyde oxidase activity (EC Number 1.17.3.2) which is thought to be distinct from AOX's activity.

SNP A3_117040611 (SEQ ID NO: 1) has the following sequence:
GGAACGAGCT CTATTCCACA TGGACAACTG TTATAACATC CCCAACATCC
[G/A]GGGCACTGG GCGGATATGC AAGACCAACC TGCCCTCCAA CACGGCCTTC A

The SNP A3_117040611 major allele is G and the minor allele is A. There was a higher frequency of the AG genotype (i.e., one copy of the major allele and one copy of the minor allele) among cats with elevated 2PY compared to its occurrence in a population with 2PY in the normal range. No cats with the GG genotype were identified, indicating that the GG genotype is lethal.

SNP A3_117041908 (SEQ ID NO: 2) has the following sequence:
CCAGGGAGAA TTGTTGGAAG AAGAGAGGGT TGTCCATAAT TCCTACCAAG
[T/C]TTGGAATAA GCTTCACTGT TCCTTTTCTG AATCAGGTAA TTGCCTTGTA T

The SNP A3_117041908 major allele is T and the minor allele is C.

SNP A3_117081913 (SEQ ID NO: 3) has the following sequence:
ACAAGAATTG TTTTTACGGG GCATGTTGAC CAGGATGTCA GCAGACCCTC
[G/A]GCGGGACTG CAGAGGGGGG CCTGGGTGTG GTCCCAGCTG TAGGGTGCGT G

The SNP A3_117081913 major allele is also G and the minor allele is also A. There was a higher frequency of the AG genotype (i.e., one copy of the major allele and one copy of the minor allele) and AA (i.e., two copies of the minor allele) among cats with elevated 2PY compared to its occurrence in a population with 2PY in the normal range.

XDH Protein (SEQ ID NO: 4) has the following
sequence:
MTADELVFFVNGKKVVEKNADPETTLLAYLRRKLGLSGTKLGCGEGGCG
ACTVMLSKYDRFQNKIVHFSANACLAPICSLHHVAVTTVEGIGSTKSRL
HPVQERIAKSHGSQCGFCTPGIVMSMYTLLRNQPEPTIEEIEDAFQGNL
CRCTGYRPILQGFRTFARDGGCCGGSGNDLNCCMNQKTDHKITLSPSLF
NPEEFTPLDPTQEPIFPPELLRLKDTPQKQLRFEGERVTWIQASTLQEL
LDLKAQDPEAKLVVGNTEIGIEMKFKNMLFPKMVCPAWIPEPVEHGPEG
ISFGASCPLSLVEKTLLDAVANLPAHQTEVFKGVLEQLRWFAGKQVKSV
ASIGGNIITASPISDLNPVFMASGAKLTIVSTGTRRTVRMDHTFFPAYR
KTLLAPEEILLSIEIPYSREGEYFSAFKQASRREDDIAKVTSGMRVLFN
PGTAQVKELALCYGGMHDRTVSALQTTRKQISNFWNEELLQNVCAGLAE
ELSLAPDAPGGMVEFRRTLTLSFFFKFYLTVLQKLGIQNSKDKCGKLDP
THASATLLFQKDPPANVQLFQEVPKGQCEEDMVGRPLPHLAAAMQASGE
AVYCDDIPRYENELSLRLVTSTRAHAKIKSIDTSEAQKVPGFVCFISAD
DVPGSNITGIGNDEMVFAKDKVTCIGHIIGAVVTDTREHAQRAAQAVRI
TYEDLPAIITIEDAIAKDSFYEPELKIEKGNLTKGFSEADNIVSGELYI
GGQEHFYLETHCTIAVPKGEAGEMELFVSTQNTTKTQSFVANMLGVPAN
RILVRVKRMGGGFGGKETRSTVVSTAVPLAAYKTGRPVRCMLDRDEDML
ITGGRHPFLARYKVGFMKTGRVVALKVEHYSNAGNTLDLSQSIMERALF
HMDNCYNIPNI[R/E]GTGRICKTNLPSNTAFRGFGGPQGMLIAEHWMS
EVAVTCGLPAEEVRRKNMYKEGDLTHFNQKLEGFTLPRCWEECLASSQY
HARKREADKFNEENCWKKRGLSIIPTK[F/L]GISFTVPFLNQAGALVH
VYTDGSVLLTHGGTEMGQGLHTKMVQVASRALKIPTSKIYISETSTNTV
PNTSPTAASVSTDINGQAVYEACQTILKRLEPFKKKNPSGSWEDWVTAA
YLDAVSLSATGFYKTPNIGYSFETNSGNPFHYFSYGVACSEVEIDCLTG
DHKNLRTDIVMDVGSSLNPAIDIGQVEGAFVQGLGLFTLEELHYSPEGS
LHTRGPSTYKIPAFGSIPSEFRVSLLRDCPNKKAIYASKAVGEPPLFLA
ASIFFAIKDAICAARAGNPDCKTKKLFQLNSPATPEKIRNACVDQFTRL
CVTGTAESCKPWSVRV

To identify a feline subject as a cat as having a higher likelihood or risk of developing CKD, a genotypic analysis using a sample from the feline subject may be undertaken to interrogate the genome for the presence of a minor allele (A) in SNP A3_117040611 (such feline having the genotype AG for that position; also referred to as the AG haplotype) or for the presence of a minor allele (A) in SNP A3_117081913 (such feline having the genotype AG or AA for that position, also referred to as the AG haplotype and the AA haplotype, respectively). In some embodiments, the genotypic analysis may interrogate the genome for both the presence of a minor allele (A) in SNP A3_117040611 (the AG haplotype) and the presence of a minor allele (A) in SNP A3_117081913 (the AG haplotype or the AA haplotype AG).

The genotypic analysis informs the need for preventative treatment and the need to monitor kidney function and health more diligently. Identifying cats with genotypes associated with or linked to higher likelihood or increased risk for developing CKD can be used in methods to treat CKD to prevent CKD or reduce the severity of disease. Identifying cats with genotypes associated with or linked to higher likelihood or increased risk for developing CKD can be used in methods to treat CKD to prevent CKD or reduce the severity of disease. Identifying cats with genotypes associated with or linked to higher likelihood or increased risk for developing CKD can be used as part of a broader health care strategy in which identified cats are monitored more closely for disease.

In some embodiments, the sample is a genomic DNA sample. In some embodiments, the sample is obtained from blood, saliva, follicle root, nasal swab or oral swab of the feline subject.

In some embodiments, methods of detecting the presence of a SNP A3_117040611 minor allele (the AG haplotype) and/or the presence of SNP A3_117081913 minor allele (the AG haplotype or the AA haplotype) in a biological sample obtained from the feline subject are provided.

In some embodiments, the biological sample is a genomic DNA sample from the feline subject using the commercially available kit such as PERFORMAgene PG-100 Oral sample collection (DNA Genotek, OraSure Technologies, Inc., Bethlehem, Pa.).

In some embodiments, SNPs are detected using methods that include at least one nucleic acid analysis technique selected from: DNA sequencing, restriction enzyme digest, polymerase chain reaction (PCR), hybridization, real-time PCR, reverse transcriptase PCR, or ligase chain reaction.

In some embodiments, SNPs are detected by performing at least one nucleic acid analysis technique selected from the group consisting of: analysis using a whole genome SNP chip; single-stranded conformational polymorphism (SSCP) assay; restriction fragment length polymorphism (RFLP); automated fluorescent sequencing; clamped denaturing gel electrophoresis (CDGE); denaturing gradient gel electrophoresis (DGGE); mobility shift analysis; restriction enzyme analysis; heteroduplex analysis; chemical mismatch cleavage (CMC); RNase protection assays; use of polypeptides that recognize nucleotide mismatches; allele-specific PCR; sequence analysis; and SNP genotyping.

In some embodiments, SNPs are detected using a method selected from the types of methods consisting of: hybridization-based methods, enzyme-based methods, post-amplification methods based on physical properties of DNA, and sequencing methods.

In some embodiments, SNPs are detected using a method selected from the types of methods consisting of: hybridization-based methods selected from the group consisting of: dynamic allele-specific hybridization, molecular beacon methods and SNP microarrays; enzyme-based methods selected from the group consisting of: restriction fragment length polymorphism (RFLP), PCR-based methods, Flap endonuclease, primer extension methods, 5′-nuclease and oligonucleotide ligation assay; post-amplification methods based on physical properties of DNA selected from the group consisting of: single strand conformation polymorphism, temperature gradient gel electrophoresis, denaturing high performance liquid chromatography, high-resolution amplicon melting, DNA mismatch-binding proteins, SNPlex, and surveyor nuclease assay; and sequencing methods

In some embodiments, SNPs are detected using a high-density array that contains genetic markers including the genetic markers for one or both SNPs.

In some embodiments, SNPs are detected using a low-density array that contains genetic markers one or both SNPs.

In some embodiments, the SNPs are detected using a high-density array containing genetic markers. Examples of arrays include the commercially available microarrays.

In some embodiments, the MassARRAY System is used in the detection of the presence of SNPs. The MassARRAY System is a non-fluorescent detection platform utilizing mass spectrometry to accurately measure PCR-derived amplicons. Mass spectrometry, coupled with end point PCR, enables highly multiplexed reactions under universal cycling conditions to provide accurate, rapid, and cost-effective analysis. The MassARRAY System offers a unique solution for targeted genetic testing with limited input material.

In some embodiments, bead array technology is used in the detection of the presence of SNPs. For example, the Illumina BeadArray technology and the Infinium HD assay (Illumina, Inc. San Diego, Calif.) may be used. In some embodiments, bead array technology is used in the detection of the presence of SNPs. The Illumina BeadArray technology is based on small silica beads that self-assemble in microwells on planar silica slides. Each bead is covered with hundreds of thousands of copies of a specific oligonucleotide that act as a capture sequence in the Infinium assay. Once the beads have self-assembled, a proprietary decoding process maps the location of every bead, ensuring that each one is individually quality controlled. The result of this manufacturing process is that every BeadChip undergoes rigorous testing to assure the highest possible quality standards. The Infinium assay can be scaled to unlimited multiplexing without compromising data quality, unlike many alternative PCR-dependent assays. The simple streamlined workflow is common across all products, no matter how many SNPs are being interrogated. Likewise, the data acquisition process and analysis are the same. The Infinium assay protocol features single-tube sample preparation and whole genome amplification without PCR or ligation steps significantly reducing labor and sample handling errors. After hybridizing unlabeled DNA samples on the Beadchip, two-step allele detection provides high call rates and accuracy. Selectivity and specificity are accomplished in two-steps. Target hybridization to bead-bound 50-mer oligos provides high selectivity while enzymatical single-base extension also incorporates a labeled nucleotide for assay readout. The staining reagent is optimized to provide a higher signal, and more balanced intensities between red and green channels. These features contribute to accuracy, high call rates and copy number data with low noise. The Infinium assay produces two-color readouts (one color for each allele) for each SNP in a genotyping study. Intensity values for each two-color channels, A and B, convey information about the allelic ratio at a single genomic locus. Typical studies incorporate values for a large number of samples (hundreds to tens of thousands) to ensure significant statistical representation. When these values are appropriately normalized and plotted distinct patterns (or clusters) emerge, in which samples have identical genotypes at an assayed locus exhibit similar signal profiles (A and B values) and aggregate in clusters. For diploid organisms, bi-allelic loci are expected to exhibit three clusters (AA, AB and BB). Genotype calls are based upon information derived from standard cluster file, which provides statistical data from a representative sample set. This enables genotypes to be called by referencing assay single intensities against known data for a given locus. Since the call accuracy is tied to the quality of the cluster data, having efficient and robust clustering algorithm is essential for accurate genotyping. The Illumina Gebtrain2 algorithm accurately and efficiently identifies cluster pattern of genotyping samples and reports summary.

SNPs may be detected using hybridization-based methods. Examples of hybridization-based methods include dynamic allele-specific hybridization, methods that employ molecular beacons, and methods that employ SNP microarrays including high-density oligonucleotide SNP arrays or low-density oligonucleotide SNP arrays. SNPs can be interrogated by hybridizing complementary DNA probes to the SNP site. In dynamic allele-specific hybridization, a genomic segment is amplified and attached to a bead through a PCR reaction with a biotinylated primer. The amplified product is then attached to a streptavidin column and washed to remove the unbiotinylated strand. An allele-specific oligonucleotide is then added in the presence of a molecule that fluoresces when bound to double-stranded DNA. The intensity is measured as temperature is increased until the melting temperature (Tm) can be determined. SNP are detected by their lower-than-expected Tm. Specifically engineered single-stranded oligonucleotide probes are used in SNP detection that uses molecular beacons. Oligonucleotides are designed in which complementary regions are at each end and a probe sequence is located in between such that the probes take on a hairpin, or stem-loop, structure in its natural, isolated state. A fluorophore is attached to one end of the probe a fluorescence quencher is attached to the other end. The fluorophore is in close proximity to the quencher when the oligo is in a hairpin configuration and the molecule does not emit fluorescence. The probe sequence is complementary to the genomic DNA used in the assay. If the probe sequence of the molecular beacon encounters its target genomic DNA during the assay, it will anneal and hybridize. The oligo will no longer assume the hairpin configuration and will fluoresce. High-density oligonucleotide SNP arrays comprise hundreds of thousands of probes arrayed on a small chip, allowing for many SNPs to be interrogated simultaneously. Several redundant probes designed to have the SNP site in several different locations as well as containing mismatches to the SNP allele are used to interrogate each SNP. The differential amount of hybridization of the target DNA to each of these redundant probes, allows for specific homozygous and heterozygous alleles to be determined.

SNPs may be detected using enzyme-based methods. A broad range of enzymes including DNA ligase, DNA polymerase and nucleases may be employed. Examples of enzyme-based methods include methods based upon restriction fragment length polymorphism (RFLP), PCR-based methods, methods that utilize Flap endonuclease; methods that utilize primer extension, methods that utilize 5′-nuclease and methods that include oligonucleotide ligation assays. RFLP methods to detect SNPs use many different restriction endonucleases to digestion a genomic sample. It is possible to ascertain whether or not the enzymes cut the expected restriction sites by determining fragment lengths through a gel assay. RFLP assays are designed to include enzymes that cut in the presence or absence of SNPs and the pattern of fragment lengths can be used to determine the presence or absence of SNPs. PCR based methods include tetra-primer amplification refractory mutation system PCR, or ARMS-PCR, and multiple qPCR reactions. Tetra-primer amplification refractory mutation system PCR, or ARMS-PCR, employs two pairs of primers to amplify two alleles in one PCR reaction. The primers are designed such that the two primer pairs overlap at a SNP location but each match perfectly to only one of the possible SNPs. Alternatively, multiple qPCR reactions can be run with different primer sets that target each allele separately. Some embodiments utilize Flap endonuclease (FEN), which is an endonuclease that catalyzes structure-specific cleavage. This cleavage is highly sensitive to mismatches and can be used to interrogate SNPs with a high degree of specificity. A FEN called cleavase is combined with two specific oligonucleotide probes, that together with the target DNA, can form a tripartite structure recognized by cleavase. The first probe, called the Invader oligonucleotide is complementary to the 3′ end of the target DNA. The last base of the Invader oligonucleotide is a non-matching base that overlaps the SNP nucleotide in the target DNA. The second probe is an allele-specific probe which is complementary to the 5′ end of the target DNA, but also extends past the 3′ side of the SNP nucleotide. The allele-specific probe will contain a base complementary to the SNP nucleotide.

Primer extension is a two-step process that first involves the hybridization of a probe to the bases immediately upstream of the SNP nucleotide followed by a ‘mini-sequencing’ reaction, in which DNA polymerase extends the hybridized primer by adding a base that is complementary to the SNP nucleotide. This incorporated base is detected and determines the SNP allele. The primer extension method is used in a number of assay formats. These formats use a wide range of detection techniques that include MALDI-TOF Mass spectrometry (see Sequenom) and ELISA-like methods. Sequenom's iPLEX SNP genotyping method, which uses a MassARRAY mass spectrometer. The flexibility and specificity of primer extension make it amenable to high throughput analysis. Primer extension probes can be arrayed on slides allowing for many SNPs to be genotyped at once. Referred to as arrayed primer extension (APEX), this technology has several benefits over methods based on differential hybridization of probes.

Illumina Incorporated's Infinium assay is an example of a whole-genome genotyping pipeline that is based on primer extension method. In the Infinium assay, over 100,000 SNPs can be genotyped. The assay uses hapten-labelled nucleotides in a primer extension reaction. The hapten label is recognized by antibodies, which in turn are coupled to a detectable signal. APEX-2 is an arrayed primer extension genotyping method which is able to identify hundreds of SNPs or mutations in parallel using efficient homogeneous multiplex PCR (up to 640-plex) and four-color single-base extension on a microarray. The multiplex PCR requires two oligonucleotides per SNP/mutation generating amplicons that contain the tested base pair. Methods that utilize 5′-nuclease include methods using Taq DNA polymerase's 5′-nuclease activity in the TaqMan assay for SNP genotyping. The TaqMan assay is performed concurrently with a PCR reaction and the results can be read in real-time as the PCR reaction proceeds. In methods that include oligonucleotide ligation assays, oligonucleotide DNA ligase catalyzes the ligation of the 3′ end of a DNA fragment to the 5′ end of a directly adjacent DNA fragment. This mechanism can be used to interrogate a SNP by hybridizing two probes directly over the SNP polymorphic site, whereby ligation can occur if the probes are identical to the target DNA. Examples of other post-amplification methods for detecting SNPs include methods based upon DNA's physical properties. Such methods first involve PCR amplification of the target DNA.

Several methods of detecting SNPs are based upon DNA's physical properties such as melting temperature and single stranded conformation. Methods that use single stranded conformation are based upon single-stranded DNA (ssDNA) that folds into a tertiary structure. The conformation is sequence dependent and most single base pair mutations will alter the shape of the structure. When applied to a gel, the tertiary shape will determine the mobility of the ssDNA, providing a mechanism to differentiate between SNP alleles. This method first involves PCR amplification of the target DNA. The double-stranded PCR products are denatured using heat and formaldehyde to produce ssDNA. The ssDNA is applied to a non-denaturing electrophoresis gel and allowed to fold into a tertiary structure. Differences in DNA sequence will alter the tertiary conformation and be detected as a difference in the ssDNA strand mobility. Temperature gradient gel electrophoresis (TGGE) or temperature gradient capillary electrophoresis (TGCE) methods are based on the principle that partially denatured DNA is more restricted and travels slower in a gel or other porous material. In another method, denaturing high performance liquid chromatography (DHPLC) uses reversed-phase HPLC to interrogate SNPs. In DHPLC, the solid phase which has differential affinity for single and double-stranded DNA. Another method used is high-resolution melting of the entire amplicon. Use of DNA mismatch-binding proteins may also be used to detect SNPs. MutS protein from Thermus aquaticus binds different single nucleotide mismatches with different affinities and can be used in capillary electrophoresis to differentiate all six sets of mismatches. SNPlex is a proprietary genotyping platform sold by Applied Biosystems. Surveyor nuclease assay uses surveyor nuclease, a mismatch endonuclease enzyme that recognizes all base substitutions and small insertions/deletions (indels), and cleaves the 3′ side of mismatched sites in both DNA strands. Sequencing technologies can also be used in SNP detection. Advances in sequencing technology allow SNP detection by sequencing more practical.

Genotyping by sequencing using next generation sequencing technologies has become a common practice. Genotyping by sequencing, also called GBS, is a method to discover single nucleotide polymorphisms (SNP) in order to perform genotyping studies, such as genome-wide association studies (GWAS). GBS uses restriction enzymes to reduce genome complexity and genotype multiple DNA samples. After digestion, PCR is performed to increase the fragment pool and then GBS libraries are sequenced using next generation sequencing technologies. With the advancement of next generation sequencing technologies such as Illumina short read sequencing by synthesis and PacBio's single molecule real time sequencing it is becoming more feasible to do GBS. In the future, development of new technologies such as nanopore single molecule sequencing may allow whole genome sequencing/genotyping.

The detection of the presence of one copy or 2 copies of a minor allele of either one or both single nucleotide polymorphisms in a biological sample obtained from the feline subject is useful to identify feline subjects with a higher likelihood or increased risk of developing CKD.

Monitoring

Genotypic analysis can inform the need for more diligent monitoring of a cat's kidney health and function and therefore be part of a method that includes identification of risk together with vigilance. In such methods, a cat with a higher likelihood or risk of developing CKD is identified using genotypic analysis of a sample from the feline subject and determining the haplotype of SNP A3_117040611, or the haplotype of SNP A3_117081913, or of both the haplotype of SNP A3_117040611 and the haplotype of SNP A3_117081913. A feline subject that has AG haplotype of SNP A3_117040611 and/or the AG or AA haplotype of the SNP A3_117081913 is monitored more closely such as by for example one or more of more frequent examination and testing. Cats identified as being at an elevated risk for CKD would be observed for clinical signs such as polyuria, polydipsia, weight loss, decreased appetite, lethargy, dehydration, vomiting, and bad breath, physical examination findings, blood test results and urinalysis. Physical examinations on cats identified as being at an elevated risk for CKD would be performed to determine palpable kidney abnormalities, evidence of weight loss, dehydration, pale mucous membranes, uremic ulcers, evidence of hypertension, i.e., retinal hemorrhages/detachment. Cats identified as being at an elevated risk for CKD may be monitored for azotemia (increased BUN and creatinine) and hyperphosphatemia. As noted above, creatinine levels increasing within the reference interval and/or SDMA levels increasing within the reference interval, persistent increased SDMA >14 μg/dL, abnormal kidney imaging, persistent renal proteinuria showing a urine protein to creatinine (UPC) ratio >0.4 in cats are consistent with a CKD diagnosis. Increased creatinine and SDMA concentrations plus USpG of <1.035 indicate a more advanced CKD diagnosis. In some embodiments, the monitoring methods comprise testing 2PY levels. In cats that present as healthy but have been identified as having an elevated risk for CKD upon detection of at least one of the genetic markers, determining 2PY levels while the cats are healthy provides a baseline level. At later time points, 2PY levels can be determined again and compared to the baseline levels. Increase in 2PY levels suggests possible kidney disease.

In some embodiments, the cat is identified as being healthy and having a higher likelihood or risk of developing CKD and 2PY levels are measured annually, twice yearly, three times yearly, four times yearly, every two months or monthly. In some embodiments, the cat is identified as being healthy and having a higher likelihood or risk of developing CKD and 2PY levels are measured twice yearly in intervals of about six months, three times yearly in intervals of about four months, or four times yearly in intervals of about three months.

In some embodiments, the cat is identified as CKD and 2PY levels are measured annually, twice yearly in intervals of about six months, three times yearly in intervals of about four months, four times yearly in intervals of about three months, every two months or monthly. In some embodiments, the cat is identified as CKD and 2PY levels are measured twice yearly in intervals of about six months, three times yearly in intervals of about four months, or four times yearly in intervals of about three months.

Treatment

Treatment methods are provided to prevent, delay onset and/or reduce severity of CKD in cats. In some embodiments, cats are test for the presence of SNP haplotype associated with increased risk and likelihood of developing CKD. Such cats that are identified as having an increased risk and likelihood of developing CKD can be treated with various methods to support kidney health. In some embodiments, such cats are fed a diet as described herein, i.e. a diet that includes effective amounts of betaine, oat beta glucan and one or more of short-chain fructo-oligosaccharide and apple pomace. In some embodiments, cats that are identified as having an increased risk and likelihood of developing CKD and being treated with methods to support kidney health such as being fed a diet that effective amounts of betaine, oat beta glucan and one or more of short-chain fructo-oligosaccharide and apple pomace, are monitored for kidney health and function as described above, including in some embodiments, by monitoring 2PY levels.

Treatment methods are provided to treat cats that have CKD. Such treatment methods are intended to reduce, ameliorate or eliminate CKD in the afflicted cat The methods comprise the step of feeding such cats a diet that comprises effective amounts of betaine, oat beta glucan and one or more of short-chain fructo-oligosaccharide and apple pomace. In some embodiments, the identification of cats with kidney disease is performed by methods that include measuring 2PY values and finding them elevated compared to a reference standard or baseline level. In some embodiments, following diagnosis, 2PY values are measured during treatment with the diet described herein to assess the cat's response to the treatment.

The treatments used to reduce the likelihood or risk of developing CKD in a feline subject identified as having a higher likelihood or risk of developing CKD and the treatments used to reduce, ameliorate or eliminate CKD in cat that are identified as having CKD comprises feeding the feline subject a diet that comprises effective amounts of betaine, oat beta glucan and one or more of short-chain fructo-oligosaccharide and apple pomace as described herein.

Kits and Reagents

Kits, reagents, other articles, and compositions useful in methods for identifying a feline subject as either being a cat with a higher likelihood or risk of developing CKD are provided. The kits, reagents, other articles, and compositions may be useful in methods that evaluate genotype and/or methods that evaluate 2PY levels. Kits, reagents, other articles, and compositions useful in methods that evaluate 2PY levels may comprise reagents useful in assays to measure such 2PY levels and may also comprise positive control samples and negative control samples.

Kits, reagents, other articles, and compositions useful methods are provided for monitoring cats identified as having an increased likelihood of developing CKD and for monitoring treatment of feline subjects identified as having CKD are provided. Such kits, reagents, other articles, and compositions useful in methods that evaluate 2PY levels include material useful in assays to measure 2PY levels and may also comprise positive control samples and negative control samples.

Compositions and Formulations

Application of the methodology outlined above comprising feeding the cat a diet that includes effective amounts of betaine, oat beta glucan and one or more of short-chain fructo-oligosaccharide and apple pomace. Cats that have been identified as being at risk, having an increased likelihood or otherwise predisposed to developing CKD, may be fed a diet that betaine, oat beta glucan and one or more of short-chain fructo-oligosaccharide and apple pomace in amounts effective to prevent, delay the onset of or reduce the severity of CDK. In some embodiments, such cats being treated using such a diet may have 2PY levels monitored as part of the treatment. Cats that have been identified as having CKD may be fed a diet that betaine, oat beta glucan and one or more of short-chain fructo-oligosaccharide and apple pomace in amounts effective to eliminate, ameliorate or reduce the severity of CDK. In some embodiments, such cats being treated using such a diet may have 2PY levels monitored as part of the treatment. A reduction in elevated 2PY levels indicates responsiveness to the treatment.

In some embodiments, the effective amounts of betaine, oat beta glucan and one or more of short-chain fructo-oligosaccharide and apple pomace are components that have been combined with other ingredients to provide a nutritionally complete diet. In some embodiments, the food product is a nutritionally complete diet for an adult feline. In a specific aspect, the food product is a nutritionally complete diet formulated for an adult domestic cat.

In some embodiments, the nutritional composition comprises 0.1-1.5% betaine, 0.1-1.5% oat beta glucan and one or more of 0.010-0.150% short-chain fructo-oligosaccharide and 1-7% apple pomace. In some embodiments, the nutritional composition comprises 0.3-0.8% betaine, 0.3-0.8% oat beta glucan and one or more of 0.025-0.075% short-chain fructo-oligosaccharide and 2.5-6% apple pomace. In some embodiments, the nutritional composition comprises 0.5% betaine, 0.586% oat beta glucan and either 0.047% short-chain fructo-oligosaccharide or 3.44% apple pomace.

In some embodiments, the compositions include food compositions is suitable for consumption by a companion animal, particularly a cat, that comprise effective amounts of betaine, oat beta glucan and one or more of short-chain fructo-oligosaccharide and apple pomace in combination with protein and/or fat and/or carbohydrate. In some embodiments, for example, in addition to betaine, oat beta glucan and one or more of short-chain fructo-oligosaccharide and apple pomace, a nutritionally complete and balanced cat food composition may comprise: from 4% to 90%, from 4% to 75%, from 5% to 75%, from 10% to 60% protein, or from 15% to 50% by weight of protein based on the total weight of the composition on a dry matter basis; from 0% to 90%, from 2% to 80%, from 5% to 75%, and from 10% to 50% by weight of carbohydrate based on the total weight of the composition on a dry matter basis; and from 2% to 60%, from 5% to 50%, and from 10% to 35% by weight of fat based on the total weight of the composition on a dry matter basis. In some embodiments, for example, in addition to betaine, oat beta glucan and one or more of short-chain fructo-oligosaccharide and apple pomace, a nutritionally complete and balanced cat food composition may further contain from 0 to 15% or from 2% to 8%, by weight of other vitamins, and minerals, antioxidants, and other nutrients, e.g. amino acids which support the nutritional needs of the animal. Vitamin C can be administered in this diet as ascorbic acid and its various derivatives thereof such as calcium phosphate salts, cholesteryl salt, 2-monophosphate, and the like which will function in a vitamin C like activity after ingesting by the pet. They can be in any form such as liquid, semisolid, solid and heat stable form.

Sources of proteins, carbohydrates, fats, vitamins, minerals, balancing agents, and the like, suitable for inclusion in the compositions, and particularly in the food products to be administered in methods provided herein, may be selected from among those conventional materials known to those of ordinary skill in the art.

In addition to an effective amount of betaine, oat beta glucan and one or more of short-chain fructo-oligosaccharide and apple pomace, in some embodiments, proteins useful as ingredients of the food compositions may comprise proteins from animal sources, such as animal proteins, including mammalian, avian protein, reptilian, amphibian, fish, invertebrate proteins and combinations thereof; e.g., from any of cattle, sheep, pig, goat, deer, rabbit, horse, kangaroo, their milk, curds, whey or blood, and internal tissues and organs such as smooth muscle, striated muscle, liver, kidney, intestine or heart; chicken including internal tissues and organs such as smooth muscle, striated muscle, liver, kidney, intestine or heart and chicken eggs, additional avian protein sources encompass turkey, goose, duck, ostrich, quail, pigeon, their eggs and internal tissues and organs such as smooth muscle, striated muscle, liver, kidney, intestine or heart; amphibian sources include frog or salamander, reptilian protein sources include alligator, lizard, turtle and snake; a fish protein sources include catfish, herring, salmon, tuna, bluefish, cod, halibut, trout, swordfish and their eggs; and an invertebrate protein sources include lobster, crab, clams, mussels or oysters, and combinations thereof, meat protein isolate, whey protein isolate, egg protein, mixtures thereof, and the like, as well as vegetable sources, such as corn gluten meal, wheat gluten, mixtures thereof, and the like.

In some embodiments, carbohydrates useful as ingredients of the food compositions may include but are not limited to, one or more of corn, whole yellow corn, grain sorghum, wheat, barley, rice, millet, brewers rice, oat groats, and polysaccharides (e.g., starches and dextrins) and sugars (e.g., sucrose, lactose, maltose, glucose, and fructose) that are metabolized for energy when hydrolyzed. Examples of additional carbohydrate sources suitable for inclusion in the compositions disclosed herein include, fruits and vegetables.

Fats useful as ingredients of the food compositions may be from any source, such as but not limited to poultry fat, beef tallow, lard, choice white grease, soybean oil, corn oil, canola oil, sunflower oil, mixtures thereof, and the like. The fat may be incorporated completely within the food composition, deposited on the outside of the food composition, or a mixture of the two methods.

In some embodiments, the compositions further include an effective amount of one or more substances selected from the group consisting of glucosamine, chondroitin, chondroitin sulfate, methylsulfonylmethane (“MSM”), creatine, antioxidants, Perna canaliculata, omega-3 fatty acids, omega-6 fatty acids and mixtures thereof.

In some embodiments, the food composition further comprises one or more amino acid such as but not limited to arginine, histidine, isoleucine, leucine, lysine, methionine (including DL-methionine, and L-methionine), phenylalanine, threonine, tryptophan, valine, taurine, carnitine, alanine, aspartate, cystine, glutamate, glutamine, glycine, proline, serine, tyrosine, and hydroxyproline.

In some embodiments, the food composition further comprises one or more fatty acids such as but not limited to lauric acid, myristic acid, palmitic acid, palmitoleic acid, margaric acid, margaroleic acid, stearic acid, oleic acid, linoleic acid, g-linolenic acid, a-linolenic acid, stearidonic acid, arachidic acid, gadoleic acid, DHGLA, arachidonic acid, eicosatetraenoic acid, EPA, behenic acid, erucic acid, docosatetraenoic acid, and DPA.

In some embodiments, the food composition further comprises one or more macro nutrients such as but not limited to moisture, protein, fat, crude fiber, ash, dietary fiber, soluble fiber, insoluble fiber, raffinose, and stachyose.

In some embodiments, the food composition further comprises one or more micro nutrients such as but not limited to beta-carotene, alpha-lipoic acid, glucosamine, chondroitin sulfate, lycopene, lutein, and quercetin.

In some embodiments, the food composition further comprises one or more minerals such as but not limited to calcium, phosphorus, potassium, sodium, chloride, iron, copper, copper, manganese, zinc, iodine, selenium, selenium, cobalt, sulfur, fluorine, chromium, boron, and oxalate.

In some embodiments, the food composition further comprises one or more other vitamins, such as but not limited to vitamin A, vitamin C, vitamin D, vitamin E, quinoa grain, thiamine, riboflavin, niacin, pyridoxine, pantothenic acid, folic acid, vitamin B12, biotin, and choline.

In some embodiments, the food composition further comprises fiber, which may be supplied from a variety of sources, including, for example, vegetable fiber sources such as cellulose, beet pulp, peanut hulls, and soy fiber.

In some embodiments, the food composition further comprises stabilizing substances, for example, substances that tend to increase the shelf life of the composition. Potentially suitable examples of such substances include, for example, preservatives, antioxidants, synergists and sequestrants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches.

In some embodiments, the food composition further comprises additives for coloring, palatability, and nutritional purposes include, for example, colorants; iron oxide, sodium chloride, potassium citrate, potassium chloride, and other edible salts; vitamins; minerals; and flavoring. The amount of such additives in a composition typically is up to 5% (dry matter basis of the composition).

Preparation of Compositions

The compositions that comprise effective amounts of betaine, oat beta glucan and one or more of short-chain fructo-oligosaccharide and apple pomace may be prepared as food products suitable for consumption by cats. These food products may be of any consistency or moisture content; i.e., the compositions may be moist, semi-moist, or dry food products. “Moist” food products are generally those with a moisture content of from 60% to 90% or greater. “Dry” food products are generally those with a moisture content of from 3% to 11%, and are often manufactured in the form of small pieces or kibbles. “Semi-moist food products generally have a moisture content of from 25% to 35%. The food products may also include components of more than one consistency, for example, soft, chewy meat-like particles or pieces as well as kibble having an outer cereal component or coating and an inner “cream” component.

In some embodiments, the food products that comprise effective amounts of betaine, oat beta glucan and one or more of short-chain fructo-oligosaccharide and apple pomace may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture is heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The compositions and food products of the present invention can also be added to or combined with food compositions before, during, or after their preparation.

In some embodiments, the food products may be prepared in a dry form using conventional processes known to those of ordinary skill in the art. Typically, dry ingredients, including dried animal protein, plant protein, grains and the like are ground and mixed together. Liquid or moist ingredients, including fats, oils, water, animal protein, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In some embodiments, the compositions are formulated so as to be easier to chew. In specific embodiments, the compositions and food products are formulated to address specific nutritional differences between species and breeds of animals, as well as one of more of the attributes of the animal. For example, cat foods, for example, are typically formulated based upon the life stage, age, size, weight, body composition, and breed.

In another embodiment, treats comprising effective amounts of betaine, oat beta glucan and one or more of short-chain fructo-oligosaccharide and apple pomace can be prepared by, for example, an extrusion or baking process similar to those described below for dry food to provide an edible product. Treats include, for example, compositions that are given to an animal to entice the animal to eat during a non-meal time. Treats may be nutritional, wherein the composition comprises one or more nutrients, and may, for example, have a composition as described above for food. Non-nutritional treats encompass any other treats that are non-toxic. Compositions can be coated onto the treat, incorporated into the treat, or both.

In another embodiment, an animal toy is provided that is a chewable or consumable toy. Such toys are typically prepared by coating any existing toy with effective amounts of betaine, oat beta glucan and one or more of short-chain fructo-oligosaccharide and apple pomace. Toys therefore include, for example, chewable toys. Contemplated toys for cats include, for example, artificial bones. In certain embodiments, the composition of the invention can form a coating on the surface of the toy or on the surface of a component of the toy, or it can be incorporated partially or fully throughout the toy, or both. A wide range of suitable toys are currently marketed. See, e.g., U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). It should be recognized that this invention contemplates both partially consumable toys (e.g., toys comprising plastic components) and fully consumable toys (e.g., rawhides and various artificial bones).

All publications mentioned herein are incorporated by reference for the purpose of describing and disclosing the materials and methodologies that are reported in the publication, which might be used in connection with the invention.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### EXAMPLES

### Example 1

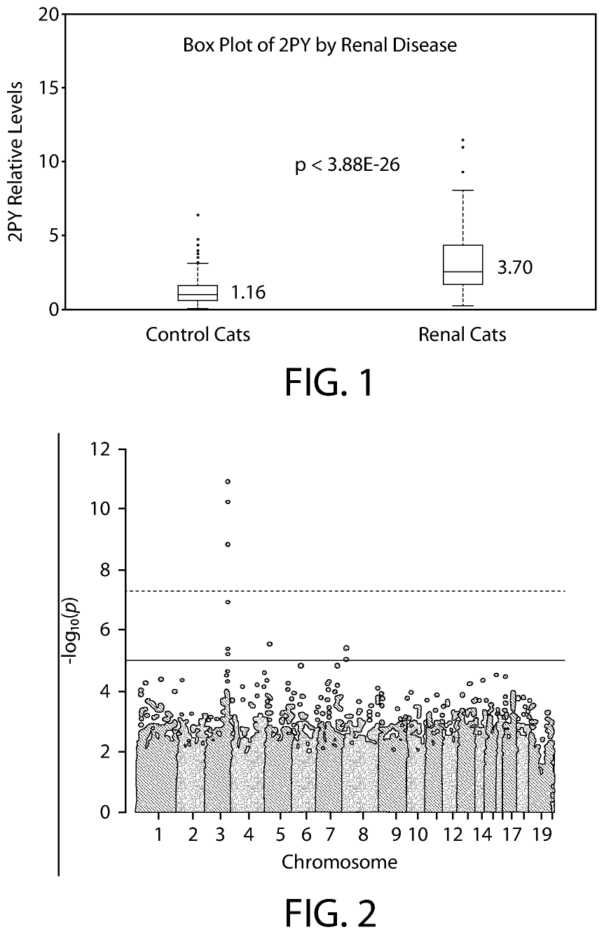

Metabolite profiling was performed on serum from a cohort of 960 cats. A large number of cats with highly elevated levels of 2PY was observed in the data set. The association between the serum levels of 2PY and a diagnosis of chronic renal disease was investigated in this cohort. Cats that had been diagnosed with chronic renal disease (n=48) were compared to an age matched healthy control group (n=350). As shown in FIG. 1, the control group was found to have an average 2PY level of 1.16 while the renal groups average was 3.70 with a p value <3.88E-26.

To determine the validity of 2PY as a potential biomarker for renal disease a logistic regression analysis between 2PY and individuals with or without a renal disease diagnosis was performed and compared that to a logistic regression with the common standard renal disease biomarker creatinine on the same cats. Data in Table 1 shows results of regression analysis of 2PY and creatine as predictors of renal disease. 2PY was found to have an odds ratio of 2.40 (p=2.40E-17) compared to 2.7 (p=6.71E-14) for creatinine, the gold standard for renal disease diagnosis. These results indicate that 2PY is on par with creatinine as a predictor of renal disease.

TABLE 1
Regression analysis of 2PY and creatine
as predictors of renal disease

| | Full model | Log 10 Full | | |
|---|---|---|---|---|
| Predictor | P value | model P value | Odds Ratio | Sample size |
| 2PY | 1.51E−17 | 16.81983893 | 2.389864218 | 425 |
| Creatinine | 6.71E−14 | 13.17336753 | 2..777618443 | 425 |

### Example 2

A genetic analysis was undertaken to gain insight into molecular mechanisms that may contribute to higher levels of 2PY and in turn drive the development of renal damage and the progression of renal disease. A QTL analysis was performed on a subset (859) of the 960 cohort that were previously genotyped on a custom iSelect Illumina genotyping array. Using this approach, a single locus with 3 SNPs exceeding genome wide significance was identified. A Manhattan plot for QTL analysis of 2PY is shown in FIG. 2. Data in Table 2 shows that there are 3 SNPs that exceed genome wide significance for 2PY associations. The significant SNPs are located on cat Chromosome A3 Base Positions 117040611 (Sequence ID:1), 117041908 (Sequence ID:2), and 117081913 (Sequence ID:3) as defined by the felCat 6.2 reference genome for cat.

TABLE 2
SNPs that exceed genome wide significance for 2PY associations

| CHR | SNP | BP | BETA | R2 | P |
|---|---|---|---|---|---|
| A3 | A3_117040611 | 117040611 | 0.6955 | 0.05684 | 1.28E−11 |
| A3 | A3_117081913 | 117081913 | 0.4372 | 0.05273 | 6.00E−11 |
| A3 | A3_117041908 | 117041908 | 0.4434 | 0.04568 | 1.46E−09 |

Paired Linkage analysis Data in Table 3 indicates that there are two independent loci in close proximity that impact serum levels of 2PY. SNP A3_117041908 and A3_117081913 are tightly linked while SNP A3_117040611 is independent of the other two.

TABLE 3
Linkage analysis indicates that SNPs
A3_117041908 and A3_117081913 are linked and
SNP A3_117040611 is an independent locus

| Marker 1 | Marker 2 | Distance in kB | EM - R Squared |
|---|---|---|---|
| A3_117040611 | A3_117041908 | 1.27 | 0.044903068 |
| A3_117040611 | A3_117081913 | 41.302 | 0.050047527 |
| A3_117041908 | A3_117081913 | 40.005 | 0.795865501 |

As shown in Table 2, genotypes at SNPs A3_117040611 and A3_117081913 have an R2 of 0.056 and 0.053, respectively, indicating that these two loci account for approximately 10% of the variance in serum 2PY levels in this cohort. Also shown in Table 2, each of these loci have a positive beta indicating that the minor allele results in an increase in the level of 2PY. Table 4 shows the mean level of 2PY for a given genotype at each of these loci. In the case of A3_117040611, there were 785 individuals with a high-quality genotype call at that SNP and a measurement of serum 2PY levels. 606 individuals were homozygous for the reference allele (G), 179 heterozygous, and no individuals were homozygous for the minor allele (A) indicating that the homozygous mutant allele may be lethal. The mean serum level of 2PY in individuals homozygous for the reference allele was 1.187 while the mean serum level for the heterozygote was almost double at 2.094. In the case of the A3_117081913 locus, there was a high-quality genotype call for 793 cats with 494 homozygous for the reference allele (G), 229 heterozygous, and 70 homozygous for the minor allele (A). The individuals homozygous for the reference allele had a mean serum level of 0.775, individuals homozygous for the minor allele had a two-fold increase in serum 2PY at 1.571, and heterozygotes had an intermediate level of 1.390, indicating an additive effect for each A allele.

TABLE 4
The number of individuals in the analysis with a given
genotype and their corresponding mean level of 2PY

| | GENOTYPE | A/A | A/G | G/G |
|---|---|---|---|---|
| A3_117040611 | COUNTS | 0 | 179 | 606 |
| | FREQUENCY | 0 | 0.2277 | 0.771 |
| | MEAN 2PY LEVEL | 0 | 2.094 | 1.187 |
| | GENOTYPE | A/A | A/G | G/G |
| A3_117081913 | COUNTS | 70 | 229 | 494 |
| | FREQUENCY | 0.08827 | 0.2888 | 0.623 |
| | MEAN 2PY LEVEL | 1.570823493 | 1.389261045 | 0.774684 |

All three significant SNPs map to the cat Xanthine Dehydrogenase/Oxidase gene (XDH) located on cat Chromosome A3:117005768-117046300. SNP A3_117040611 results in an A instead of a G at base pair 11704611. This transition results in arginine 894 being replaced with a glutamine. Furthermore, SNP A3_117041908 is a transition of a T to a C resulting in phenylalanine 1004 being replaced with a leucine. It is possible that these two mis-sense mutations explain the two independent effects within the XDH locus on serum levels of 2PY. (XDH Protein Sequence ID:4)

The presence of at least one minor allele in either SNP A3_117040611 or SNP A3_117081913, which are each within the XDH locus, has been identified and associated with an increased likelihood or risk of CKD. Accordingly, with respect to SNP A3_117040611, the major allele is G and the G/G genotype is the homozygous major allele genotype. The minor allele is A. The A/G genotype is the heterozygous genotype that includes one copy of the minor allele. The A/A genotype is the homozygous minor allele genotype. As noted above, no cats were identified with the homozygous minor allele genotype (A/A) of SNP A3_117040611. Cats identified with a heterozygous genotype minor allele of SNP A3_117040611 (i.e. the A/G genotype) are identified as having an elevated or increased likelihood or risk of CKD and candidates for treatment intervention, such as modified diet, to reduce risk. With respect to SNP A3_117081913, the major allele is G and the G/G genotype is the homozygous major allele genotype. The minor allele is A. The A/G genotype is the heterozygous genotype that includes one copy of the minor allele; the A/A genotype is the homozygous minor allele genotype that includes two copies of the minor allele. Cats identified with a heterozygous genotype of SNP A3_117081913 (i.e., the A/G genotype) and those with the homozygous minor allele genotype of SNP A3_117081913 (i.e., the A/A genotype) are identified as having an elevated or increased likelihood or risk of CKD and candidates for treatment intervention, such as modified diet, to reduce risk.

The genotypic differences translate to detectable phenotypic differences. Cats that have the AG genotype of SNP A3_117040611 and cats that have either AG or AA genotype of the SNP A3_117081913 have elevated levels of 2PY compared to cats that have the GG genotype of the SNPs. Accordingly, in addition to or instead of determining genotype in order to identify a cat as having a higher likelihood or risk of developing CKD, the phenotypic difference, i.e., 2PY level, can also be used to identify whether a feline subject has a higher likelihood or risk of developing 2PY.

No association was detected between 2PY levels in cat and the AOX gene locus. M-methyl-nicotinamide may be metabolized by XDH in the cat or there is little variance in AOX activity in the cat and the mis-sense mutations introduced by SNPs SNP A3_117040611 and SNP A3_117041908 alter the function of XDH so that N-methyl-nicotinamide acts as a substrate and is oxidized to 2PY. Either way cats with these genotypes have the capability of producing higher levels of 2PY which in turn can accelerate kidney damage contributing to the progression of chronic kidney disease.

### Example 3

A diet that reduces the serum level of 2PY may be beneficial to cats, especially cats that are genetically predisposed to produce higher than average levels of 2PY. Cats with high levels of 2PY may progress toward renal failure at a faster rate than cats with lower levels of 2PY. By lowering the level of 2PY through dietary means the progression of renal disease may be slowed. To this end the following two diet(s) (diet 1 and diet 2) were developed and were effective to achieve significant reductions in plasma concentration of 2PY. Both diets have similar ingredient profiles with the baseline food (BSL) except that diet 1 is supplemented with 0.5% betaine, 0.586% oat beta glucan and 0.047% short-chain fructo-oligosaccharide. Diet 2 is supplemented with 0.5% betaine, 0.586% oat beta glucan and 3.44% apple pomace. The nutrient profiles of the foods are shown in Table 5.

TABLE 5
Nutrient profiles of the baseline food (BSL), Diet 1 and Diet 2

| | BSL | Diet 1 | Diet 2 |
|---|---|---|---|
| Moisture | 5.47 | 5.34 | 5.97 |
| Protein | 27.6 | 28 | 27.3 |
| Fat | 19.9 | 19.5 | 19.5 |
| Ash | 4.5 | 4.42 | 4.59 |
| Crude fiber | 1.3 | 2.0 | 2.1 |
| Insoluble fiber | 4.0 | 3.4 | 5.1 |
| Soluble fiber | 1.3 | 0.8 | 1.6 |
| Atwater Energy (kcal/kg) | 4101 | 4063 | 4032 |

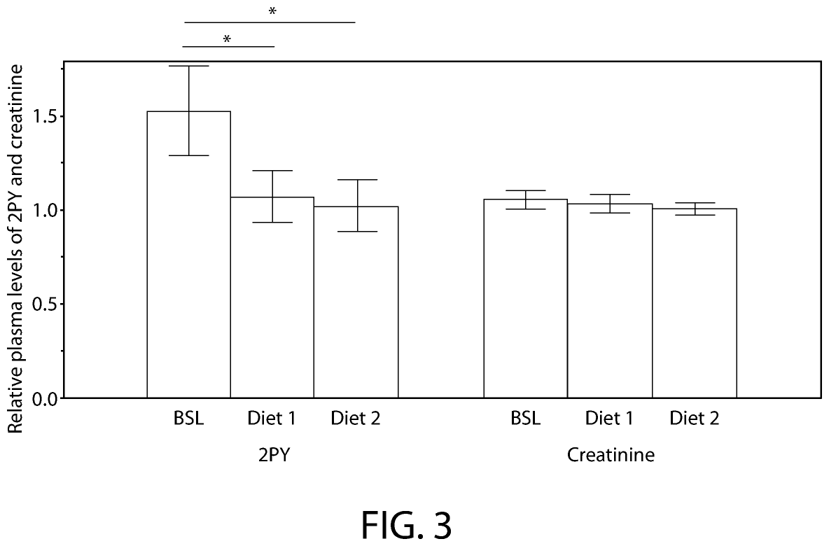

A study conducted on 10 renal and 10 healthy cats showed that renal cats have significantly higher relative plasma concentrations of 2PY (P=0.0083) and creatinine (P=0.01) compared to healthy cats. After all cats were fed the baseline food (BSL) for 30 days, half of the healthy and renal groups were fed diet 1 for 30 days before they switched to diet 2 for 30 days. The other half of the healthy and renal groups were fed diet 2 first before they switched to diet 1. Blood samples were collected at the end of each 30 days feeding period to measure plasma metabolites including 2PY and creatinine. The average relative plasma levels of creatinine were not statistically different between the three foods. However, diet 1 (P=0.002) and diet 2 (P=0.001) led to a significant reduction in plasma concentration of 2PY from BSL (FIG. 3). The relative concentrations of 2PY between Diet 1 and Diet 2 were not significantly different (P>0.05). Cats that had similar creatinine levels after they consumed the 3 foods (Table 6). The results suggest that the inclusion of betaine, oat beta glucan and sc-FOS or apple pomace leads to a significant reduction in plasma levels of 2PY, which is elevated in renal cats compared to healthy cats. The fact that creatinine levels stayed the same while 2PY levels were reduced significantly within 30 days suggests the importance of measuring 2PY as a biomarker responding faster to dietary interventions slowing the progression of CKD in cats.

TABLE 6
Relative plasma levels of 2PY and creatinine after cats consumed
the baseline food (BSL), Diet 1 or Diet 2, each for 30 days

| | BSL | Diet 1 | Diet 2 |
|---|---|---|---|
| 2PY | 1.57 | 1.09 | 1.04 |
| Creatinine | 1.02 | 1.0 | 1.01 |

1. A method of detecting the presence of one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject, the method comprising:
analyzing a biological sample obtained from the feline subject to detect one copy or 2 copies of a minor allele of one or more single nucleotide polymorphisms selected from the group consisting of SNP A3_117040611, SNP A3_117041908 and SNP A3_117081913 and a single nucleotide polymorphism in linkage disequilibrium with one or more thereof.

2. The method of claim 1 wherein the single nucleotide polymorphisms selected from the group consisting of SNP A3_117040611 and SNP A3_117081913.

3. A method of identifying a feline subject as being at an increased likelihood of developing cat kidney disease comprising:
analyzing a biological sample obtained from the feline subject for the presence of one copy or 2 copies of a minor allele of two or more single nucleotide polymorphisms selected from the group consisting of SNP A3_117040611, SNP A3_117041908 and SNP A3_117081913 and a single nucleotide polymorphism in linkage disequilibrium with one or more thereof in a feline subject;
wherein the presence of one copy or 2 copies of the minor allele of two or more single nucleotide polymorphisms selected from the group consisting of SNP A3_117040611, SNP A3_117041908 and SNP A3_117081913 indicates that the feline subject has an increased likelihood of developing cat kidney disease within its lifetime.

4. The method of claim 3 wherein the single nucleotide polymorphisms selected from the group consisting of SNP A3_117040611 and SNP A3_117081913.

5. The method of claim 3,
wherein:
the sample is analyzed by performing DNA sequencing, restriction enzyme digest, polymerase chain reaction (PCR), hybridization, real-time PCR, reverse transcriptase PCR, or ligase chain reaction.

6. The method of claim 5 wherein the single nucleotide polymorphisms selected from the group consisting of SNP A3_117040611 and SNP A3_117081913.

7. The method of claim 3 comprising the additional step of quantifying the level of 2PY present in a blood sample from the feline subject.

8. The method of claim 3 wherein the sample is a genomic DNA sample.

9. The method of claim 3 wherein the sample is obtained from blood, saliva, follicle root, nasal swab or oral swab of the feline subject.

10. The method of claim 3 wherein the sample is analyzed by performing at least one nucleic acid analysis technique selected from: DNA sequencing, restriction enzyme digest, polymerase chain reaction (PCR), hybridization, real-time PCR, reverse transcriptase PCR, or ligase chain reaction.

11. The method of claim 3 wherein the sample is analyzed by performing at least one nucleic acid analysis technique selected from: analysis using a whole genome SNP chip, single-stranded conformational polymorphism (SSCP) assay, restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis, heteroduplex analysis, chemical mismatch cleavage (CMC), RNase protection assays, use of polypeptides that recognize nucleotide mismatches, allele-specific PCR, sequence analysis, and SNP genotyping.

12. The method of claim 3 wherein the sample is analyzed by performing at least one nucleic acid analysis technique selected from: hybridization-based methods, enzyme-based methods, post-amplification methods based on physical properties of DNA, and sequencing methods.

13. The method of claim 3 wherein the sample is analyzed by performing at least one nucleic acid analysis technique selected from: hybridization-based methods selected from the group consisting of dynamic allele-specific hybridization, molecular beacon methods and SNP microarrays; enzyme-based methods selected from the group consisting of restriction fragment length polymorphism (RFLP), PCR-based methods, Flap endonuclease, primer extension methods, 5′-nuclease and oligonucleotide ligation assay; post-amplification methods based on physical properties of DNA selected from the group consisting of single strand conformation polymorphism, temperature gradient gel electrophoresis, denaturing high performance liquid chromatography, high-resolution amplicon melting, DNA mismatch-binding proteins, SNPlex, and surveyor nuclease assay; and sequencing methods.

14. A method of monitoring the health of feline subject identified as having an increased likelihood of developing CKD comprising
identifying a feline subject as being at an increased likelihood of developing cat kidney disease according to claim 3;
quantifying at two or more time points the level of 2PY present in a blood sample from the feline wherein an elevation of 2PY levels over time indicates that the feline subject is developing kidney disease.

15. A method of monitoring the health of feline subject comprising
quantifying at two or more time points the level of 2PY present in a blood sample from the feline subject identified as having cat kidney disease wherein an elevation of 2PY levels over time indicates that the feline subject is developing kidney disease.

16. A method of delaying onset and severity of cat kidney disease in a feline subject identified as having an increased likelihood of developing CKD comprising
detecting in a biological sample from the feline subject, the presence of one copy or 2 copies of a minor allele of one or more single nucleotide polymorphisms according to claim 1; and
administering to the feline subject a composition comprising an effective amount of: betaine, oat beta glucan and one or more of short-chain fructo-oligosaccharide and apple pomace.

17. A method of delaying onset and severity of cat kidney disease in a feline subject suspected of having an increased likelihood of developing CKD comprising
administering to the feline subject a composition comprising an effective amount of: betaine, oat beta glucan and one or more of short-chain fructo-oligosaccharide and apple pomace.

18. The method of claim 16 wherein the composition is a nutritional composition that is fed to the feline subject.

19. The method of claim 16 wherein the nutritional composition comprises 0.1-1.5% betaine, 0.1-1.5% oat beta glucan and one or more of 0.010-0.150% short-chain fructo-oligosaccharide and 1-7% apple pomace.

20. The method of claim 16 wherein the nutritional composition comprises 0.5% betaine, 0.586% oat beta glucan and either 0.047% short-chain fructo-oligosaccharide or 3.44% apple pomace.

21. A method of monitoring the treatment of feline subject identified as having cat kidney disease and being treated for cat kidney disease comprising
quantifying the level of 2PY present in a blood sample from the feline subject identified as having cat kidney disease wherein the blood sample was obtained prior to treating the feline subject for cat kidney disease;
administering treatment to the feline subject;
quantifying the level of 2PY present in a blood sample from the feline subject obtained after treating the feline subject for cat kidney disease;
wherein a reduction of 2PY levels after treatment indicates that the feline subject is responding to the treatment.

22. A solid support having affixed thereon a collection of isolated nucleic acids wherein:
each nucleic acid of the collection is 20-200 nucleotides in length;
the collection includes 5000 different isolated nucleic acids or less; and the collection includes a minor allele and a major allele for one or more single nucleotide polymorphisms selected from the group consisting of SNP A3_117040611, SNP A3_117041908 and SNP A3_117081913,
and a single nucleotide polymorphism in linkage disequilibrium with one or more thereof in a feline subject.

23. The solid support of claim 22 wherein the collection includes 4000, 3000, 2000, 1000, 750, 500, 400, 300, 200, 100, 75, 50 different isolated nucleic acids or less.

24. A food composition comprising an effective amount of betaine, oat beta glucan and one or more of short-chain fructo-oligosaccharide and apple pomace.

25. The food composition of claim 24 comprising 0.1-1.5% betaine, 0.1-1.5% oat beta glucan and one or more of 0.010-0.150% short-chain fructo-oligosaccharide and 1-7% apple pomace.

26. The food composition of claim 24 comprising 0.5% betaine, 0.586% oat beta glucan and either 0.047% short-chain fructo-oligosaccharide or 3.44% apple pomace.

27. The food composition of claim 24 wherein the food composition is a feline food composition.


SEQUENCE LISTING (4 sequences)

SEQ ID NO: 1 - DNA, 101 bp, Felis catus
  source 1..101: mol_type = genomic DNA
ggaacgagctctattccacatggacaactgttataacatccccaacatccrgggcactgggcggatatgcaagaccaacctgccctccaacacggccttca

SEQ ID NO: 2 - DNA, 101 bp, Felis catus
  source 1..101: mol_type = genomic DNA
ccagggagaattgttggaagaagagagggttgtccataattcctaccaagyttggaataagcttcactgttccttttctgaatcaggtaattgccttgtat

SEQ ID NO: 3 - DNA, 101 bp, Felis catus
  source 1..101: mol_type = genomic DNA
acaagaattgtttttacggggcatgttgaccaggatgtcagcagaccctcrgcgggactgcagaggggggcctgggtgtggtcccagctgtagggtgcgtg

SEQ ID NO: 4 - protein, 881 aa, Felis catus
  VARIANT 444: note = X is R or E
  VARIANT 554: note = X is F or L
ALCYGGMHDRTVSALQTTRKQISNFWNEELLQNVCAGLAEELSLAPDAPGGMVEFRRTLTLSFFFKFYLTVLQKLGIQNSKDKCGKLDPTHASATLLFQKDPPANVQLFQEVPKGQCEEDMVGRPLPHLAAAMQASGEAVYCDDIPRYENELSLRLVTSTRAHAKIKSIDTSEAQKVPGFVCFISADDVPGSNITGIGNDEMVFAKDKVTCIGHIIGAVVTDTREHAQRAAQAVRITYEDLPAIITIEDAIAKDSFYEPELKIEKGNLTKGFSEADNIVSGELYIGGQEHFYLETHCTIAVPKGEAGEMELFVSTQNTTKTQSFVANMLGVPANRILVRVKRMGGGFGGKETRSTVVSTAVPLAAYKTGRPVRCMLDRDEDMLITGGRHPFLARYKVGFMKTGRVVALKVEHYSNAGNTLDLSQSIMERALFHMDNCYNIPNIXGTGRICKTNLPSNTAFRGFGGPQGMLIAEHWMSEVAVTCGLPAEEVRRKNMYKEGDLTHFNQKLEGFTLPRCWEECLASSQYHARKREADKFNEENCWKKRGLSIIPTKXGISFTVPFLNQAGALVHVYTDGSVLLTHGGTEMGQGLHTKMVQVASRALKIPTSKIYISETSTNTVPNTSPTAASVSTDINGQAVYEACQTILKRLEPFKKKNPSGSWEDWVTAAYLDAVSLSATGFYKTPNIGYSFETNSGNPFHYFSYGVACSEVEIDCLTGDHKNLRTDIVMDVGSSLNPAIDIGQVEGAFVQGLGLFTLEELHYSPEGSLHTRGPSTYKIPAFGSIPSEFRVSLLRDCPNKKAIYASKAVGEPPLFLAASIFFAIKDAICAARAGNPDCKTKKLFQLNSPATPEKIRNACVDQFTRLCVTGTAESCKPWSVRV